SEQUENCE LISTING (6 sequences)

SEQ ID NO: 1 - DNA, 912 bp, HEMA1
atggctgctcaaggctggtccatgctcctgctggctgtccttaacctaggcatcttcgtccgtccctgtgacactcaagagctacgatgtctgtgtattcaggaacactctgaattcattcctctcaaactcattaaaaatataatggtgatattcgagaccatttactgcaacagaaaggaagtgatagcagtcccaaaaaatgggagtatgatttgtttggatcctgatgctccatgggtgaaggctactgttggcccaattactaacaggttcctacctgaggacctcaaacaaaaggaatttccaccggcaatgaagcttctgtatagtgttgagcatgaaaagcctctatatctttcatttgggagacctgagaacaagagaatatttccctttccaattcgggagacctctagacactttgctgatttagctcacaacagtgataggaattttctacgggactccagtgaagtcagcttgacaggcagtgatgcctaaaagccactcatgaggcaaagagtttcaaggaagctctcctcctggagttttggcgttctcattcttatactctattcccgcgttagtctggtgtatggatctatgagctctcttttaatattttattataaatgttttatttacttaacttcctagtgaatgttcacaggtgactgctcccccatccccatttcttgatattacatataatggcatcatatacccctttattgactgacaaactactcagattgcttaacattttgtgcttcaaagtcttatcccactccactatgggctgttacagagtgcatctcggtgtagagcaaggctccttgtcttcagtgccccagggtgaaatacttctttgaaaaattttcattcatcagaraatctgaaataaatatt

SEQ ID NO: 2 - protein, 167 aa, HEMA1
MAAQGWSMLLLAVLNLGIFVRPCDTQELRCLCIQEHSEFIPLKLIKNIMVIFETIYCNRKEVIAVPKNGSMICLDPDAPWVKATVGPITNRFLPEDLKQKEFPPAMKLLYSVEHEKPLYLSFGRPENKRIFPFPIRETSRHFADLAHNSDRNFLRDSSEVSLTGSDA

SEQ ID NO: 3 - DNA, 667 bp, HEMA2
cgaggtgatcataaactcgcccatcgtcctgcgctacaagaccccctacttcaaagcctccgcccgcgtggtcatgccccccatcccccgccacgagacctgggtggtgggctggattcaggcgtgcaatcagatggagttcttcaacacctacagcgacctgggcatgtcaagctgggaactgcctgacttgagggaagggagagtaaaagccatcagtgactcagatggggtgagctacccttggtacgggaacaccacagaaactgtgaccctggttggcccaccaacaagatctccaggttctccgtcagcataatgacaacttctaccccagtgtgacatgggcagtgcctgtgagtgacagcaatgtgccactgctcacaagaatcaagagagaccaaagtttcacgacctggctggtggccatgaacaccaccacaaaggagaagatcattctgcagaccatcaagtggaggatgagggtggacattgaagtggaccctcttcagctcttggggcagcgggcccggctggtgggcaggactcagcaggagcagccccggatcctgagccggatggaacccatcccccctaatgcactagtgaaacccaatgcccaatgatgccaggtcctcatgtgggggcccagcggggccctctgttg

SEQ ID NO: 4 - protein, 62 aa, HEMA2
RGDHKLAHRPALQDPLLQSLRPRGHAPHPPPRDLGGGLDSGVQSDGVLQHLQRPGHVKLGTA

SEQ ID NO: 5 - protein, 208 aa, HEMA2
EVIINSPIVLRYKTPYFKASARVVMPPIPRHETWVVGWIQACNQMEFFNTYSDLGMSSWELPDLREGRVKAISDSDGVSYPWYGNTTETVTLVGPTNKISRFSVSMNDNFYPSVTWAVPVSDSNVPLLTRIKRDQSFTTWLVAMNTTTKEKIILQTIKWRMRVDIEVDPLQLLGQRARLVGRTQQEQPRILSRMEPIPPNALVKPNAQ

SEQ ID NO: 6 - protein, 67 aa, HEMA1
EFPPAMKLLYSVEHEKPLYLSFGRPENKRIFPFPIRETSRHFADLAHNSDRNFLRDSSEVSLTGSDA

(19) United States
(12) Patent Application Publication
(10) Pub. No.: US 2005/0042655 A1
(43) Pub. Date: Feb. 24, 2005

(54) Novel hematopoietic regulatory factors and methods of use thereof

(75) Inventors: David Lewin, New Haven, CT (US); Richard Shimkets, West Haven, CT (US); Lawrence Lasky, Sausalito, CA (US); Bruce Taillon, Middletown, CT (US); Steven Gold, Branford, CT (US)

Correspondence Address: MERCHANT & GOULD PC, P.O. BOX 2903, MINNEAPOLIS, MN, 55402-0903, US

(73) Assignees: Genentech Corporation, South San Francisco, CA (US); Curagen Corporation, New Haven, CT (US)

(21) Appl. No.: 10/886,160

(22) Filed: Jul. 6, 2004

Related U.S. Application Data
(63) Continuation of application No. 09/640,636, filed on Aug. 17, 2000, now abandoned.
(60) Provisional application No. 60/149,830, filed on Aug. 19, 1999.

(51) Int. Cl. C12Q 1/68

(52) U.S. Cl. 435/6

(57) ABSTRACT

Disclosed are novel hematopioetic modulating nucleic acid sequences and polypeptides. Also disclosed are methods in which the nucleic acids and polypeptides are utilized in the detection and treatment of hematopoietic disorders.

### RELATED U.S. APPLICATIONS

This application claims priority to U.S. Ser. No. 60/149,830 filed Aug. 19, 1999, which is incorporated herein by reference in its entirety.

### FIELD OF THE INVENTION

The invention relates generally to nucleic acids and polypeptides, as well as vectors, host cells, antibodies and recombinant methods for producing the polypeptides and polynucleotides.

### BACKGROUND OF THE INVENTION

Hematopoiesis is the process of blood cell formation. Hematopoiesis is a complex process requiring interplay between various cellular signals. Understanding the regulation of hematopoiesis is important in several human diseases and conditions, e.g., anemia, leukemia and cancer.

Hematopoietic cells include e.g., erythrocytes, lymphocytes, and cells of myeloid lineage. These cell types all arise from the same pluripotent stem cells. In an adult, hematopoiesis occurs in the bone marrow where stem cells divide infrequently to produce more stem cells (self-renewel) and various committed progenitor cells. It is the committed progenitor cells that will in response to specific regulator factors produce a hematopoietic cell. These regulatory factors are primarily produced by the surrounding stromal cells and in other tissues and include, for example, colony-stimulating factors (CSFs), erythropoietin (EPO), interleukin 3 (IL3), granulocyte/macrophage CSF (GM-CSF), granulocyte CSF (G-CSF), macrophage CSF (M-CSF), and STEEL factor.

### SUMMARY OF THE INVENTION

The invention is based, in part, on the discovery of novel polynucleotide sequences encoding hematopoietic regulatory factors. These novel sequences are referred to herein as “HEMA” nucleic acid and polypeptides. These novel nucleic acids were identified by differential gene expression analysis of endothelial cell lines.

In one aspect, the present invention provides an isolated nucleic acid molecule (SEQ ID NO: 1 and 3,) that encodes a hematopoietoic regulatory related polypeptide (HEMA), or fragment, homolog, analog or derivative thereof. The nucleic acid can include, e.g., a nucleic acid sequence encoding a polypeptide that is at least 80% identical to the polypeptides of FIG. 2 (SEQ ID NO:2 and SEQ ID NO: 4). The nucleic acid can be, e.g., a genomic DNA fragment, or it can be a cDNA molecule.

In another aspect the invention provides a chimeric polypeptide which includes a chemokine linked to a hematopoietic modulating sequence. In one aspect of the invention the hematopoietic modulating sequence comprises the amino acids of SEQ ID NO: 6. The chimeric polypeptides are referred to herein as “CHEMA” polypeptides.

In various aspects, the invention includes methods of assesing hematopoietic status, methods of diagnosing and treating hematopiotic disorders. For example, in one aspect, the invention provides a method of assessing hematopoietic status by providing a test cell population that includes one or more cells capable of expressing one or more HEMA nucleic acids. Levels of expression of one or more sequences in a test cell population are then compared to the levels of expression of the corresponding nucleic acids in a reference cell population. The reference cell population contains cells whose hematopoietic status is known, i.e., it is known whether the reference cell has the ability to modulate the differentiation and/or proliferation of hematopoietic stem cells.

In another aspect, the invention provides a method of diagnosing or determining susceptibility to hematopoietic disorders, e.g., anemia, cancer, and leukemia. The method includes providing from the subject a cell population comprising a cell capable of expressing one or more HEMA nucleic acids, and comparing the expression of the nucleic acid sequences to the expression of the nucleic acid sequences in a reference cell population that includes cells from a subject not suffering from a hematopoietic disorder.

In various other aspects, the invention provides methods of regulating hematopoeisis by modulating hematopoietoic stem cell migration, proliferation and differentiation. For example, in one aspect the invention provides a method for identifying agents that modulate hematopoeisis by contacting a HEMA or CHEMA polypeptide with the compound and determining whether the compound modifies activity of the HEMA or CHEMA polypeptide, binds to the HEMA or CHEMA polypeptide, or binds to a nucleic acid molecule encoding a HEMA or CHEMA polypeptide.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based in part on the discovery of differences in expression patterns of multiple nucleic acid sequences between culture stromal cells derived from the aorta-gonad-mesonephros region of murine embroyos.

The differentially expressed nucleic acids were identified by comparing nucleic acid expression differences between the endothelial cell lines DAS1044, DAS104-8 and YS CL72. The endothelial cell line b-End 3 was used as a control. Genes whose transcript levels varied between the cell lines were identified using GENECALLING™ differential expression analysis as described in U.S. Pat. No. 5,871,697 and in Shimkets et al., Nature Biotechnology 17:798-803 (1999). The contents of these patents and publications are incorporated herein by reference in their entirety.

Over 2000 gene fragments were initially found to be differentially expressed in the DAS 104-4 DAS 104-8 and YS CL72 cell lines. Genes fragments whose expression levels appeared to increase or decrease more than 5-fold compared to control cells were selected for further analysis. An unlabeled oligonucleotide competition assay as described in Shimkets et al., Nature Biotechnology 17:198-803 was used to verify the identity of differentially expressed sequences.

Forty single copy nucleic acid sequences whose expression levels differed between DAS 104-4 DAS 104-8 and YS CL72 cell lines were chosen for further characterization. These sequences are referred to herein as HEMA 1-40. A summary of the HEMA sequences analyzed is presented in Table 1.

Two sequences (HEMA: 1-2) represent novel murine genes. The 38 other sequences identified have been previously described.

For some of the novel sequences (i.e., HEMA 1-2), a cloned sequence is provided along with one or more additional sequence fragments (e.g., ESTs or contigs) which contain sequences identical to, or substantially identical to, the cloned sequence. Also provided is a consensus sequence which includes a composite sequence assembled from the cloned and additional fragments. For a given HEMA sequence, its expression can be measured using any of the associated nucleic acid sequence in the methods described herein. For previously described sequences (HEMA:3-40), database accession numbers are provided. This information allows for one of ordinary skill in the art to deduce information necessary for detecting and measuring expression of the HEMA nucleic acid sequences.

The nucleic acids discussed herein include the following:

TABLE 1

| | | Effect on Transcript Level | | | HEMA | |
|---|---|---|---|---|---|---|
| Gene Name | GenBank Acc# | DAS 104-8 | YS CL72 | DAS 104-4 | Assignment | SEQ ID NO: |
| 09.04 Novel | | | | | | |
| pg_mm_d21095: mouse similar to | | −1.3 | −2.5 | 5.7 | 1 | 1 and 2 |
| Norway rat d21095 Rat mRNA for | | | | | | |
| CINC-2 beta, complete cds. [N] | | | | | | |
| cgmmg0y044.4_7: | | 3.1 | — | 13 | 2 | 3, 4 and 5 |
| 01.01.04 Regulators | | | | | | |
| Hif1a: Mus musculus hypoxia-inducible | af003695 | 5.6 | −3.1 | 6.4 | 3 | |
| factor 1 alpha (Hif1a) mRNA, | | | | | | |
| NAB2: Mus musculus NGFI-A binding | u47543 | 5.3 | 7.2 | 4.5 | 4 | |
| protein 2 (NAB2) mRNA, complete cds. | | | | | | |
| TSC-22: Mouse EST similar to human | uemm_32857_0 | 5.1 | 3.7 | 4.3 | 5 | |
| TSC-22 protein (TGFB STIMULATED | | | | | | |
| CLONE 22 HOMOLOG) | | | | | | |
| 01.05.01 Proteolysis | | | | | | |
| APP: Mouse mRNA for amyloid A4 beta | uemm_369_0 | −2.5 | 4.3 | −32 | 6 | |
| precursor (protease nexin II) | | | | | | |
| 02.01.02 Growth Factors | | | | | | |
| TGF-beta: Mouse transforming growth | m13177 | −4 | −12 | −7.4 | 7 | |
| factor beta mRNA (TGF-beta), complete | | | | | | |
| 02.01.04 Chemokines | | | | | | |
| Osteopontin: Mus musculus osteopontin | j04806 | −3.5 | — | 9.9 | 8 | |
| mRNA, complete cds. | | | | | | |
| 02.02.01 Tyrosine Kinase Receptors | | | | | 9 | |
| FLT1: Mus musculus mRNA for flt-1, | d88689 | −6.9 | −5.6 | −35 | 10 | |
| complete cds. Also known as VEGF | | | | | | |
| Receptor-1; Fms/Kit/PDGF receptor | | | | | | |
| family-related tyrosine kinase receptor | | | | | | |
| MYK1: Mus musculus Balb/c eph-related | u06834 | −9.2 | −4.7 | −9.4 | 11 | |
| receptor protein tyrosine kinase, myk1 | | | | | | |
| 02.04.02 Beta Subunits | | | | | | |
| GNB2-RS1: Mouse mRNA for G protein | d29802 | 14 | 25 | 5.5 | 12 | |
| beta subunit homologue, complete cds. | | | | | | |
| Also known as p205. | | | | | | |
| 02.11 Kinases | | | | | 13 | |
| PKM2: Mouse mRNA for pyruvate kinase | d38379 | 3.1 | 4.9 | 3.4 | 14 | |
| M. | | | | | | |
| PKM1 and PKM2: Mouse mRNA for | uemm_1954_1 | 3.1 | 4.9 | 6.3 | 15 | |
| pyruvate kinase M1/M2 type | | | | | | |
| 02.11.01 Serine/Threonine Kinases | | | | | | |
| m15424: M15424 Myeloproliferative | m15424 | 15 | 7.2 | 14 | 16 | |
| sarcoma virus v-mos oncogene, | | | | | | |
| 02.13.04 Calpactins | | | | | | |
| ANX11: Mus musculus annexin XI | u65986 | 2.9 | 2.1 | 5.3 | 17 | |
| (ANX11) mRNA, complete cds. Also | | | | | | |
| known as calcyclin-associated annexin 50 | | | | | | |
| 03.03.06.02 Apoptosis Inhibition | | | | | | |
| uemm_168_0: Mouse mRNA for sulfated | uemm_168_0 | 3.7 | −5.8 | 4 | 18 | |
| glycoprotein-2/clustrin/apolipoprotein J | | | | | | |
| mRNA, complete cds SIM complement- | | | | | | |
| associated protein SP-40, 40 | | | | | | |
| PEA-15: M. musculus mRNA for | x86694 | 3.1 | 3.9 | 4.1 | 19 | |
| astrocytic phosphoprotein, PEA-15. | | | | | | |
| 04.01.02.03 Microsomal | | | | | | |
| Omega Oxidation | | | | | | |
| SCD2: Mouse stearoyl-CoA | m26270 | 5.8 | 6 | 5 | 20 | |
| desaturase | | | | | | |
| 04.06.06 Sugar/Nucleotide | | | | | | |
| Biosynthesis and Conversions | | | | | | |
| af007267: Mus musculus | af007267 | 3.6 | 3.4 | 4.9 | 21 | |
| phosphomannomutase Sec53p homolog | | | | | | |
| mRNA, complete cds. | | | | | | |
| cgmmg010273.5_1: Novel UDP | Cgmmg010273.5_1 | 4.2 | — | 5.1 | 22 | |
| Glucuronosyltransferase [N] | | | | | | |
| cgmm10c0104_5: Mouse similar | Cgmm10c0104_5 | 4.6 | 5.7 | 4.4 | 23 | |
| to worms and yeast “similar to | | | | | | |
| phosphomannomutase” [N] | | | | | | |
| 04.07.02 Folic Acid | | | | | | |
| pg_mm_m59861: mouse similar | pg_mm_m59861 | 14 | — | 49 | 24 | |
| to Norway rat m59861 Rattus norvegicus | | | | | | |
| 10-formyltetrahydrofolate dehydrogenase | | | | | | |
| mRNA, [N] | | | | | | |
| 04.11 Metabolite | | | | | | |
| Storage/Transport Proteins | | | | | | |
| pg_mm_gbh_AB012130: | pg_mm_gbh_{—} | 4 | — | 5.8 | 25 | |
| mouse similar to human gbh_AB012130 | x12609 | | | | | |
| H. sapiens mRNA SBC2 for sodium | | | | | | |
| bicarbonate cotransporter 2. [N] | | | | | | |
| 04.11.01.03 | | | | | | |
| Steroids/Hormones | | | | | | |
| cgmmp0t0197.7_5: Mouse | Cgmmp0t0197.7_5 | 2.9 | — | 4.7 | 26 | |
| similar to human Prostaglandin | | | | | | |
| Transporter (PGT) and rat matrin F/G. [N] | | | | | | |
| pg_mm_m64862: mouse similar | pg_mm_m64862 | 2.3 | — | 4.6 | 27 | |
| to Norway rat m64862 Rat matrin F/G | | | | | | |
| mRNA, complete cds. [N] | | | | | | |
| 04.11.02.01 Anions | | | | | | |
| j04036: Mus musculus band 3- | j04036 | 1.9 | 6.4 | 6 | 28 | |
| related protein mRNA, complete | | | | | | |
| 05.01.01.02 Structural Arm: | | | | | | |
| Intermediate Filaments | | | | | | |
| Desmin: Mouse desmin mRNA | uemm_18347_0 | 1.6 | — | 4.6 | 29 | |
| SIM desmin 0.0 | | | | | | |
| 06.02.01.01 Immune System | | | | | | |
| Component | | | | | | |
| C3: Mouse complement | j00367 | 3.5 | 4.3 | 4.2 | 30 | |
| component C3 gene, 5′ end. | | | | | | |
| 06.02.01.03 Receptors | | | | | | |
| CD14: Mouse CD14 mRNA for | x13333 | 2.5 | −3.6 | 4.9 | 31 | |
| myelid cell-specific leucine-rich | | | | | | |
| glycoprotein. | | | | | | |
| 08.02.01 ER/Pre-Golgi | | | | | | |
| Transfer Proteins | | | | | | |
| cgmmh0a0148.5_1: Mouse sec61 | cgmmh0a0148.5_1 | 5.4 | 12 | 11 | 32 | |
| homolog similar to cgmm37870_0 [N] | | | | | | |
| 09 Unknown Function | | | | | | |
| cgmmh0a0205.4_1: Mouse | cgmmh0a0205.4_1 | — | — | 4.7 | 33 | |
| similar to human and canine DVS27. [N] | | | | | | |
| pg_mm_u06713: mouse similar | pg_mm_u06713 | 15 | 8.3 | 37 | 34 | |
| to Norway rat u06713 Rattus norvegicus | | | | | | |
| Sprague-Dawley SM-20 mRNA, complete | | | | | | |
| cds. [N] | | | | | | |
| retinal gene 4: M. musculus DNA | y14422 | 4.5 | 2.1 | 8.8 | 35 | |
| for retinal protein. | | | | | | |
| 09.01 Known Genes | | | | | | |
| Psuedogene-Pgk1: Mus musculus | m23961 | −1.8 | 3.2 | 10 | 36 | |
| phosphoglycerate kinase (Pgk1-ps1) | | | | | | |
| processed pseudogene | | | | | | |
| u63133_0: Mus musculus C-type | u63133_0 | 30 | 40 | 22 | 37 | |
| ecotropic endogenous retrovirus, complete | | | | | | |
| mRNAsequence. | | | | | | |
| 09.01.02 Unassociated | | | | | | |
| Itm2B/E25B: Mus musculus | uemm_3350_0 | 5.8 | 1.7 | 5.7 | 38 | |
| integral membrane protein 2B | | | | | | |
| (Itm2b/E25B). [N] | | | | | | |
| x17124: Mouse DNA for virus- | x17124 | 3.4 | — | 8.6 | 39 | |
| like (VL30) retrotransposon BVL-1 - Mus | | | | | | |
| musculus, 5447 bp. | | | | | | |
| 09.02 Putative Homologies | | | | | | |
| uemm_8033_0: Mouse similar to | uemm_8033_0 | 4.1 | 4.7 | 4.3 | 40 | |
| human sarcoma amplified sequence (SAS) | | | | | | |
| [N] | | | | | | |

Below follows additional discussion of nucleic acid sequences whose expression is differentially expressed in cell lines DAS104-4, DAS104-8 and YS CL72.

HEMA1

A HEMA1 nucleic acid and polypeptide according to the invention includes the nucleic acid and encoded polypeptide sequence of pg_mm_d21095.

The predicted open reading frame codes a secreted protein that has a predicted molecular weight of 17 kD. Analysis of the HEMA1 polypepide demonstrates that the protein was homologous to several chemokines of the CXC (alpha) family. As with many chemokines in the CXC family, this novel protein contains an ELR (Glu-Leu-Arg) sequence near its amino terminus, a feature which has previously been shown to be required for receptor binding. In contrast to other CXC chemokines, this novel protein also contained an extended carboxy-terminal domain of approximately 65 amino acids (SEQ ID NO:. 6) Due to the highly unusual carboxy terminal extension of the protein and its structural homology to the chemokine family of proteins, HEMA1 was named WECHE (WEird CHEmokine).

The nucleic acid encoding for HEMA1 is localized on mouse chromosome 5 in a position we estimate to be 51 cM offset from the centromere based on comparing our mapping data to values in the composite map in the Mouse Genome Database.

Northern and PCR analyses demonstrate high level of expression of HEMA1 in the adult lung, 10.5 day old yolk sac and in AGM regions of 10.5 day old mice.

The similarity of HEM 1 polypeptides to these previously described chemokines demonstrates that the HEMA1 nucleic acids, polypeptides, antibodies and related compounds of the invention may be used to treat, prevent or diagnose a variety of hematopoietic disorders, e.g anemia, leukemia and other cancers. In addition, the HEMA 1 nucleic acids and polypeptides can also be used to identify novel agents that modulate these disorders.

The HEMA1 nucleic acid and encoded polypeptide has the following sequence:

| | | |
|---|---|---|
| 1 | atggctgctc aaggctggtc catgctcctg ctggctgtcc ttaacctagg catcttcgtc | (SEQ ID NO: 1) |
| 61 | cgtccctgtg acactcaaga gctacgatgt ctgtgtattc aggaacactc tgaattcatt | |
| 121 | cctctcaaac tcattaaaaa tataatggtg atattcgaga ccatttactg caacagaaag | |
| 181 | gaagtgatag cagtcccaaa aaatgggagt atgatttgtt tggatcctga tgctccatgg | |
| 241 | gtgaaggcta ctgttggccc aattactaac aggttcctac ctgaggacct caaacaaaag | |
| 301 | gaatttccac cggcaatgaa gcttctgtat agtgttgagc atgaaaagcc tctatatctt | |
| 361 | tcatttggga gacctgagaa caagagaata tttccctttc caattcggga gacctctaga | |
| 421 | cactttgctg atttagctca caacagtgat aggaattttc tacgggactc cagtgaagtc | |
| 481 | agcttgacag gcagtgatgc ctaaaagcca ctcatgaggc aaagagtttc aaggaagctc | |
| 541 | tcctcctgga gttttggcgt tctcattctt atactctatt cccgcgttag tctggtgtat | |
| 601 | ggatctatga gctctctttt aatattttat tataaatgtt ttatttactt aacttcctag | |
| 661 | tgaatgttca caggtgactg ctcccccatc cccatttctt gatattacat ataatggcat | |
| 721 | catatacccc tttattgact gacaaactac tcagattgct taacattttg tgcttcaaag | |
| 781 | tcttatccca ctccactatg ggctgttaca gagtgcatct cggtgtagag caaggctcct | |
| 841 | tgtcttcagt gccccagggt gaaatacttc tttgaaaaat tttcattcat cagaraatct | |
| 901 | gaaataaata tt | |

| | |
|---|---|
| MAAQGWSMLLLAVLNLGIFVRPCDTQELRCLCIQEHSEFIPLKLIKNIMVIFETIYCNRKEVIAVPKNGSMICLDPDAPWVKATVGPI | (SEQ ID NO: 2) |
| TNRFLPEDLKQKEFPPAMKLLYSVEHEKPLYLSFGRPENKRIFPFPIRETSRHFADLAHNSDRNFLRDSSEVSLTGSDA | |

HEMA2

A HEMA2 nucleic acid and polypeptide according to the invention includes the nucleic acid and encoded polypeptide sequence of cgmmg0y044.4.

Analysis of the nucleic acid sequence revealed the presence of two open reading frames.

HEMA2 is upregulated in both DAS-104-4 and DAS-104-8 as compared to the control, suggesting a role in differentiation and proliferation of hematopoietic stem cells. In addition, HEMA2 nucleic acid has 95% identity to an EST from an endometrium adenocarcinoma cell line. Thus, HEMA2 may be used as an agent to affect fertility. HEMA3 also has similarity to clone N78469 from a multiple sclerosis library. Thus, HEMA2 may also be used for diagnosis and/or treatment of multiple sclerosis. HEMA2 is 82% identical and 89% similar to pufferfish sequence CNS03HI5

The HEMA2 nucleic acid and encoded polypeptides has the following sequence:

| | |
|---|---|
| CGAGGTGATCATAAACTCGCCCATCGTCCTGCGCTACAAGACCCCCTACTTCAAAGCCTCCGCCCGCGTGGTCATGCCCCCCATCCCC | (SEQ ID NO: 3) |
| CGCCACGAGACCTGGGTGGTGGGCTGGATTCAGGCGTGCAATCAGATGGAGTTCTTCAACACCTACAGCGACCTGGGCATGTCAAGCT | |
| GGGAACTGCCTGACTTGAGGGAAGGGAGAGTAAAAGCCATCAGTGACTCAGATGGGGTGAGCTACCCTTGGTACGGGAACACCACAGA | |
| AACTGTGACCCTGGTTGGCCCACCAACAAGATCTCCAGGTTCTCCGTCAGCATAATGACAACTTCTACCCCAGTGTGACATGGGCAGT | |
| GCCTGTGAGTGACAGCAATGTGCCACTGCTCACAAGAATCAAGAGAGACCAAAGTTTCACGACCTGGCTGGTGGCCATGAACACCACC | |
| ACAAAGGAGAAGATCATTCTGCAGACCATCAAGTGGAGGATGAGGGTGGACATTGAAGTGGACCCTCTTCAGCTCTTGGGGCAGCGGG | |
| CCCGGCTGGTGGGCAGGACTCAGCAGGAGCAGCCCCGGATCCTGAGCCGGATGGAACCCATCCCCCCTAATGCACTAGTGAAACCCAA | |
| TGCCCAATGATGCCAGGTCCTCATGTGGGGGCCCAGCGGGGCCCTCTGTTG | |
| RGDHKLAHRPALQDPLLQSLRPRGHAPHPPPRDLGGGLDSGVQSDGVLQHLQRPGHVKLGTA | (SEQ ID NO: 4) |
| EVIINSPIVLRYKTPYFKASARVVMPPIPRHETWVVGWIQACNQMEFFNTYSDLGMSSWELPDLREGRVKAISDSDGVSYPWYGNTTE | (SEQ ID NO: 5) |
| TVTLVGPTNKISRFSVSMNDNFYPSVTWAVPVSDSNVPLLTRIKRDQSFTTWLVAMNTTTKEKIILQTIKWRMRVDIEVDPLQLLGQR | |
| ARLVGRTQQEQPRILSRMEPIPPNALVKPNAQ | |

Chimeric Polypeptides Including a Chemokine Domain and a Hematopoietic Modulating Domain

In another aspect the invention provides a chimeric polypetide, that includes a first and second domain. These chimeric polypeptides are referred to herein as “CHEMA” polpypeptides. The first domain includes a chemokine. The chemokine can be any known chemokine, e.g., CXC or a CC chemokine. The second domain includes a hematopoietic modulating sequence linked by a covalent bond, e.g. peptide bond, to the first domain. The first and second domains can occur in any order in the peptide, and the peptide can include one or more of each domain. The hematopoietic modulating sequence may be linked either to the N-terminal or the C-terminal end of chemokine domain.

A hematopoietic modulating sequence is any sequence of amino acids that modulates hematopoiesis. Thus, the hematopoietic modulating sequence can, for example, increase or decrease hematopoietic stem cell and endothelial cell differentiation or proliferation. For example, the hematopoietic modulating sequence may include sequences from HEMA1. In one embodiment the hematopoietic modulating sequence comprises some or all of the amino acid sequence: EFPPAMKLLYSVEHEKPLYLSFGRPENKRIFPFPIRETSRHFADLAHNSDRNFLRDSSEVSLTGSDA (SEQ ID NO: 6)

The chemokine can be a single (i.e., continuous) amino acid sequence present in the chemokine sequence. Alternatively it can be two or more amino acid sequences, which are present in the chemokine protein, but in the naturally-occurring protein are separated by other amino acid sequences. The amino acid sequence of naturally-occurring chemokine protein can be modified, for example, by addition, deletion and/or substitution of at least one amino acid present in the naturally-occurring chemokine protein, to produce modified chemokine protein.

An HEMA chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An HEMA-encoding nucleic acid can be cloned into such an expression vector such that the fusion moiety is linked in-frame to the HEMA protein.

The chemokine and the hematopoietic modulating sequence can also be linked by chemical coupling in any suitable manner known in the art.

The invention also includes pharmaceutical compositions comprising CHEMA polypeptides.

General Screening and Diagnostic Methods Using HEMA Sequences

Several of the herein disclosed methods relate to comparing the levels of expression of one or more HEMA nucleic acids in a test and reference cell populations. The sequence information disclosed herein, coupled with nucleic acid detection methods known in the art, allow for detection and comparison of the various HEMA transcripts. In some embodiments, the HEMA nucleic acids and polypeptide correspond to nucleic acids or polypeptides which include the various sequences (referenced by SEQ ID NOs) disclosed for each HEMA nucleic acid sequence.

In its various aspects and embodiments, the invention includes providing a test cell population which includes at least one cell that is capable of expressing one or more of the sequences HEMA 1-40, or any combination of HEMA sequences thereof. By “capable of expressing” is meant that the gene is present in an intact form in the cell and can be expressed. Expression of one, some, or all of the HEMA sequences is then detected, if present, and, preferably, measured. Using sequence information provided by the database entries for the known sequences, or the sequence information for the newly described sequences, expression of the HEMA sequences can be detected (if expressed) and measured using techniques well known to one of ordinary skill in the art. For example, sequences within the sequence database entries corresponding to HEMA sequences, or within the sequences disclosed herein, can be used to construct probes for detecting HEMA RNA sequences in, e.g., northern blot hybridization analyses or methods which specifically, and, preferably, quantitatively amplify specific nucleic acid sequences. As another example, the sequences can be used to construct primers for specifically amplifying the HEMA sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

For HEMA sequences whose polypeptide product is known, expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the HEMA sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803.

In various embodiments, the expression of 2, 3, 4, 5, 6, 7,8, 9, 10, 15, 20, 25, 28, 30, 35, or all of the sequences represented by HEMA 1-40 are measured. If desired, expression of these sequences can be measured along with other sequences whose expression is known to be altered according to one of the herein described parameters or conditions.

The reference cell population includes one or more cells capable of expressing the measured HEMA sequences and for which the compared parameter is known, e.g., hematopoietic status. By “hematopoietic status” is meant that is known whether the reference cell has the ability to modulate the differentiation and/or proliferation of hematopoietic stem cells. A hematopoietic stem cell is any cell that has the potential of differentiating into a hematopoietic cell, e.g. lymphoid, erythroid or myloid cell. Preferably, the hematopoietic cell is derived from the bone marrow or fetal liver.

Whether or not comparison of the gene expression profile in the test cell population to the reference cell population reveals the presence, or degree, of the measured parameter depends on the composition of the reference cell population. For example, if the reference cell population is composed of cells that support hematopoietic stem cell differentiation, a similar gene expression level in the test cell population and a reference cell population indicates the test cell population supports hematopoietic stem cell differentiation.

In various embodiments, a HEMA sequence in a test cell population is considered comparable in expression level to the expression level of the HEMA sequence in the reference cell population if its expression level varies within a factor of less than or equal to 2.0 fold from the level of the HEMA transcript in the reference cell population. In various embodiments, a HEMA sequence in a test cell population can be considered altered in levels of expression if its expression level varies from the reference cell population by more than 2.0 fold from the expression level of the corresponding HEMA sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

In some embodiments, the test cell population is compared to multiple reference cell populations. Each of the multiple reference populations may differ in the known parameter. Thus, a test cell population may be compared to a first reference cell population supporting hematopoietic stem cell differentiation, as well as a second reference population known supporting hematopoietic stem cell proliferation.

The test cell population can be any number of cells, i e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., hematopoietic cell. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (e.g., hematopoietic status, diagnostic, or therapeutic claims) is known.

The subject is preferably a mammal. The mammal can be, e.g., a human, non-human primate, mouse, rat, dog, cat, horse, or cow.

Assessing Hematopoietic Status

Expression of some of the HEMA sequences described herein is correlated with hematopoietic status. Thus, in one aspect, the invention provides a method of assessing hematopoietic status in a subject. Hematopoietic status refers to the ability of a cell to modulate the differentiation and/or proliferation of hematopoietic stem cells.

The method includes providing one or more test cell populations from the subject that includes cells capable of expressing one or more nucleic acid sequences homologous to those listed in Table 1 as HEMA. The sequences need not be identical to sequences including HEMA, as long as the sequence is sufficiently similar that specific hybridization can be detected. Preferably, the cell includes sequences that are identical, or nearly identical to those identifying the HEMA nucleic acids shown in Table 1.

Expression of the sequences is compared to a reference cell population. In general, any reference cell population can be used, as long as the hematopoietic status of the cells in the reference cell population is known. Comparison can be performed on test and reference samples measured concurrently or at temporally distinct times. An example of the latter is the use of compiled expression information, e.g., a sequence database, which assembles information about expression levels of known sequences in cells whose hematopoietic status is known.

In some embodiments, the reference cell population is made up substantially, or preferably exclusively, of hematopoietic stem cells. Example of reference cells are the stromal cell lines DAS 104-8 and DAS 104-4 and the endothelial cell line YS CL72. Expression of HEMA sequences similar to the HEMA expression pattern of DAS 104-8 indicates that the cell population has a hematopoietic status that supports the differentiation of hematopoietic stem cells. Whereas, expression of HEMA sequences similar to the HEMA expression pattern of DAS 104-4 indicates that the cell population has a hematopoietic status that supports the proliferation, i.e. self renewal of hematopoietic stems cells

Methods of Diagnosing or Determining the Susceptibility to a Hematopoietic Disorder.

The invention further provides a method of diagnosing or determining the susceptibility of a hematopoietic disorder. A disorder is diagnosed by examining the expression of one or more HEMA nucleic acid sequences from a test population of cells from a subject suspected of having the disorder.

The hematopoietic disorder can be any disorder of the hematopoietic system, e.g., anemia, leukemia and lymphomas.

Expression of one or more of the HEMA nucleic acid sequences, e.g. HEMA: 140 is measured in the test cell population and is compared to the expression of the sequences in the reference cell population. The reference cell population contains at least one cell whose disease status (i.e., the reference cell population is from an anemic subject) is known. If the reference cell population contains cells that have a disorder, then a similarity in expression between HEMA sequences in the test population and the reference cell population indicates the subject has the hematopoietic disorder. A difference in expression between HEMA sequences in the test population and the reference cell population indicates the reference cell population does not have the hematopoietic disorder.

Methods of Treating Hematopoietic Disorders

Also included in the invention is a method of treating, i.e., preventing or delaying the onset of a hematopoietic disorder in a subject by administering to the subject an agent which modulates the expression or activity of one or more nucleic acids selected from the group consisting of HEMA. “Modulates” is meant to include increased or decreased expression or activity of the HEMA nucleic acids. Preferably, modulation results in alteration of the expression or activity of the HEMA genes or gene products in a subject to a level similar or identical to a subject not suffering from the hematopoietic disorder.

The hematopoietic disorder can be any of the disorders described herein, e.g., anemia, cancer, or leukemia. The subject can be, e.g., a human, a rodent such as a mouse or rat, or a dog or cat.

The herein described HEMA nucleic acids, polypeptides, antibodies, agonists, and antagonists when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the Therapeutic can be delivered in a controlled release system including, e.g.: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Polynucleotides of the present invention can also be used for gene therapy. Gene therapy refers to therapy that is performed by the administration of a specific nucleic acid to a subject. Delivery of the Therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g., Goldspiel, et al., 1993. Clin Pharm 12:488-505.

Cells may also be cultured ex vivo in the presence of therapeutic agents or proteins of the present invention in order to proliferate or to produce a desired effect on or activity in such cells. Treated cells can then be introduced in vivo for therapeutic purposes.

Assessing Efficacy of Treatment of a Hematopoietic Disorder in a Subject

The differentially expressed HEMA sequences identified herein also allow for the course of treatment of a pathophysiology to be monitored. In this method, a test cell population is provided from a subject undergoing treatment for a hematopoietic disorder. If desired, test cell populations can be taken from the subject at various time points before, during, or after treatment. Expression of one or more of the HEMA sequences, e.g., HEMA: 1-40, in the cell population is then measured and compared to a reference cell population which includes cells whose pathophysiologic state is known. Preferably, the reference cells have not been exposed to the treatment.

If the reference cell population contains cells not exposed to the treatment and not suffering from the disorder, then a difference in expression between HEMA sequences in the test population and this reference cell population indicates the treatment is not efficacious. However, a similarity in expression between HEMA sequences in the test cell population and the reference cell population described above indicates that the treatment is efficacious By “efficacious” is meant that the treatment leads to a decrease in the pathophysiology in a subject. When treatment is applied prophylactically, “efficacious” means that the treatment retards or prevents a pathophysiology. For example, if the hematopoietic disorder is anemia, an “efficacious” treatment is one that increases red blood cell production in a subject.

Efficaciousness can be determined in association with any known method for treating the particular pathophysiology.

Promoting the Migration of Hematopoietic Stem Cells

The invention also provided a method for promoting the migration of hematopoietic stem cells. The method includes contacting a hematopoietic stems cell with one or more of the HEMA or a CHEMA polypeptides of the invention in a amount sufficient to promote migration. Preferably, the cell is contacted with HEMA 1.

The hematopoietic stem cell can be any cell that has the potential of differentiating into a hematopoietic cell. Preferably the cell is a fetal liver cell. More preferably the cell is a bone marrow cell. The cell can be any number of cells, i.e., one or more cells, and can be provided in vitro, in vivo, or ex vivo.

Inhibiting Proliferation and Differentiation

The invention also provides a method of inhibiting proliferation or differentiation of a cell. The cell can be a hematopoietic stem cell or an endothelial cell. The method includes contacting a cell with one or more of the HEMA or a CHEMA polypeptides of the invention in an amount sufficient to inhibit proliferation or differentiation. Preferably, the cell is contacted with HEMA1.

Identifying Agents that Modulate Hematopoeisis

Also included in the invention are methods of identifying agents that modulate hematopoiesis. One method includes contacting one or more HEMA or CHEMA polypeptides with a test agent and detecting a complex between the test agent and the polypeptide. A presence of a complex indicates that the test agent modulates hematopoiesis. Absence of a complex indictes that the test agent does not modulate hematopoiesis.

In another method agents that modulate hemaptoieis are identified by providing a hematopoietic stem cell and contacting the cell with one or more HEMA or CHEMA polypeptides and a test agent. Proliferation or differentiation of a hematopoietic stem cell in the presence of the polypeptide and test agent is compared to the proliferation or differentiation of a hematopoietic stem cell in the presence of the polypeptide and absence of the test agent. An alteration in proliferation or differentiation of the hematopoietic stem cell in the presence of the polypeptide and test agent compared to the proliferation or differentiation of the hematopoietic stem cell in the presence of the polypeptide and absence of the test agent indicates that the test agent modulates hematopoiesis.

By “modulate hematopoiesis” is meant that the test agent either increases or decreases the hematopoietic stem cell's ability to proliferate or differentiate into lymphoid, myloid or erythroid cells.

A test agent can be, e.g. peptides, peptidomimetics, small molecules or other drugs.

Methods of Modulating the Activity of HEMA Proteins

The invention provides a method for identifying modulators, i.e., candidate or test compounds or agents (e.g., peptides, peptidomimetics, small molecules or other drugs) that bind to HEMA proteins or have a stimulatory or inhibitory effect on, for example, HEMA expression or HEMA activity.

In one embodiment, the invention provides assays for screening candidate or test compounds which bind to or modulate the activity of the membrane-bound form of a HEMA protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci USA. 87:6378-6382; Felici (1991) J Mol Biol 222:301-310; Ladner above.).

In one embodiment, an assay is a cell-based assay in which a cell which expresses a membrane-bound form of HEMA protein, or a biologically active portion thereof, on the cell surface is contacted with a test compound and the ability of the test compound to bind to a HEMA protein determined. The cell, for example, can be of mammalian origin or a yeast cell. Determining the ability of the test compound to bind to the HEMA protein can be accomplished, for example, by coupling the test compound with a radioisotope or enzymatic label such that binding of the test compound to the HEMA protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of HEMA protein, or a biologically active portion thereof, on the cell surface with a known compound which binds HEMA to form an assay mixture, contacting the assay mixture with a test compound, and determining the ability of the test compound to interact with a HEMA protein, wherein determining the ability of the test compound to interact with a HEMA protein comprises determining the ability of the test compound to preferentially bind to HEMA or a biologically active portion thereof as compared to the known compound.

In another embodiment, an assay is a cell-based assay comprising contacting a cell expressing a membrane-bound form of HEMA protein, or a biologically active portion thereof, on the cell surface with a test compound and determining the ability of the test compound to modulate (e.g., stimulate or inhibit) the activity of the HEMA protein or biologically active portion thereof. Determining the ability of the test compound to modulate the activity of HEMA or a biologically active portion thereof can be accomplished, for example, by determining the ability of the HEMA protein to bind to or interact with a HEMA target molecule. As used herein, a “target molecule” is a molecule with which a HEMA protein binds or interacts in nature, for example, a molecule on the surface of a cell which expresses a HEMA interacting protein, a molecule on the surface of a second cell, a molecule in the extracellular milieu, a molecule associated with the internal surface of a cell membrane or a cytoplasmic molecule. A HEMA target molecule can be a non-HEMA molecule or a HEMA protein or polypeptide of the present invention. In one embodiment, a HEMA target molecule is a component of a signal transduction pathway that facilitates transduction of an extracellular signal (e.g. a signal generated by binding of a compound to a membrane-bound HEMA molecule) through the cell membrane and into the cell. The target, for example, can be a second intercellular protein that has catalytic activity or a protein that facilitates the association of downstream signaling molecules with HEMA.

Determining the ability of the HEMA protein to bind to or interact with a HEMA target molecule can be accomplished by one of the methods described above for determining direct binding. In one embodiment, determining the ability of the HEMA protein to bind to or interact with a HEMA target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a HEMA-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a HEMA protein or biologically active portion thereof with a test compound and determining the ability of the test compound to bind to the HEMA protein or biologically active portion thereof. Binding of the test compound to the HEMA protein can be determined either directly or indirectly as described above. In one embodiment, the assay comprises contacting the HEMA protein or biologically active portion thereof with a known compound which binds HEMA to form an assay mixture, contacting the assay mixture with a test compound, and determining the ability of the test compound to interact with a HEMA protein, wherein determining the ability of the test compound to interact with a HEMA protein comprises determining the ability of the test compound to preferentially bind to HEMA or biologically active portion thereof as compared to the known compound.

In another embodiment, an assay is a cell-free assay comprising contacting HEMA protein or biologically active portion thereof with a test compound and determining the ability of the test compound to modulate (e.g. stimulate or inhibit) the activity of the HEMA protein or biologically active portion thereof. Determining the ability of the test compound to modulate the activity of HEMA can be accomplished, for example, by determining the ability of the HEMA protein to bind to a HEMA target molecule by one of the methods described above for determining direct binding. In an alternative embodiment, determining the ability of the test compound to modulate the activity of HEMA can be accomplished by determining the ability of the HEMA protein further modulate a HEMA target molecule. For example, the catalytic/enzymatic activity of the target molecule on an appropriate substrate can be determined as previously described.

In yet another embodiment, the cell-free assay comprises contacting the HEMA protein or biologically active portion thereof with a known compound which binds HEMA to form an assay mixture, contacting the assay mixture with a test compound, and determining the ability of the test compound to interact with a HEMA protein, wherein determining the ability of the test compound to interact with a HEMA protein comprises determining the ability of the HEMA protein to preferentially bind to or modulate the activity of a HEMA target molecule.

The cell-free assays of the present invention are amenable to use of both the soluble form or the membrane-bound form of HEMA. In the case of cell-free assays comprising the membrane-bound form of HEMA, it may be desirable to utilize a solubilizing agent such that the membrane-bound form of HEMA is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either HEMA or its target molecule to facilitate separation of complexed from uncomplexed forms of one or both of the proteins, as well as to accommodate automation of the assay. Binding of a test compound to HEMA, or interaction of HEMA with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-HEMA fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or HEMA protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of HEMA binding or activity determined using standard techniques.

Other techniques for immobilizing proteins on matrices can also be used in the screening assays of the invention. For example, either HEMA or its target molecule can be immobilized utilizing conjugation of biotin and streptavidin. Biotinylated HEMA or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with HEMA or target molecules, but which do not interfere with binding of the HEMA protein to its target molecule, can be derivatized to the wells of the plate, and unbound target or HEMA trapped in the wells by antibody conjugation. Methods for detecting such complexes, in addition to those described above for the GST-immobilized complexes, include immunodetection of complexes using antibodies reactive with the HEMA or target molecule, as well as enzyme-linked assays that rely on detecting an enzymatic activity associated with the HEMA or target molecule.

In another embodiment, modulators of HEMA expression are identified in a method wherein a cell is contacted with a candidate compound and the expression of HEMA mRNA or protein in the cell is determined. The level of expression of HEMA mRNA or protein in the presence of the candidate compound is compared to the level of expression of HEMA mRNA or protein in the absence of the candidate compound. The candidate compound can then be identified as a modulator of HEMA expression based on this comparison. For example, when expression of HEMA mRNA or protein is greater (statistically significantly greater) in the presence of the candidate compound than in its absence, the candidate compound is identified as a stimulator of HEMA mRNA or protein expression. Alternatively, when expression of HEMA mRNA or protein is less (statistically significantly less) in the presence of the candidate compound than in its absence, the candidate compound is identified as an inhibitor of HEMA mRNA or protein expression. The level of HEMA mRNA or protein expression in the cells can be determined by methods described herein for detecting HEMA mRNA or protein.

In yet another aspect of the invention, the HEMA proteins can be used as “bait proteins” in a two-hybrid assay or three hybrid assay (see, e.g., U.S. Pat. No. 5,283,317, Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with HEMA (“HEMA-binding proteins” or “HEMA-bp”) and modulate HEMA activity. Such HEMA-binding proteins are also likely to be involved in the propagation of signals by the HEMA proteins as, for example, upstream or downstream elements of the HEMA pathway.

The two-hybrid system is based on the modular nature of most transcription factors, which consist of separable DNA-binding and activation domains. Briefly, the assay utilizes two different DNA constructs. In one construct, the gene that codes for HEMA is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a HEMA-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with HEMA.

This invention further pertains to novel agents identified by the above-described screening assays and uses thereof for treatments as described herein.

Methods of Detecting HEMA Proteins

The invention also provides a method for detecting the presence or absence of HEMA in a biological sample. The method includes obtaining a biological sample from a test subject and contacting the biological sample with a compound or an agent capable of detecting HEMA protein or nucleic acid (e.g., mRNA, genomic DNA) that encodes HEMA protein such that the presence of HEMA is detected in the biological sample. An agent for detecting HEMA mRNA or genomic DNA is a labeled nucleic acid probe capable of hybridizing to HEMA mRNA or genomic DNA. The nucleic acid probe can be, for example, a full-length HEMA nucleic acid, such as the nucleic acid of SEQ ID NO: 1 and 3, or a portion thereof, such as an oligonucleotide of at least 15, 30, 50, 100, 250 or 500 nucleotides in length and sufficient to specifically hybridize under stringent conditions to HEMA mRNA or genomic DNA. Other suitable probes for use in the diagnostic assays of the invention are described herein.

An agent for detecting HEMA protein is an antibody capable of binding to HEMA protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect HEMA mRNA, protein, or genomic DNA in a biological sample in vitro as well as in vivo. For example, in vitro techniques for detection of HEMA mRNA include Northern hybridizations and in situ hybridizations. In vitro techniques for detection of HEMA protein include enzyme linked immunosorbent assays (ELISAs), Western blots, immunoprecipitations and immunofluorescence. In vitro techniques for detection of HEMA genomic DNA include Southern hybridizations. Furthermore, in vivo techniques for detection of HEMA protein include introducing into a subject a labeled anti-HEMA antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting HEMA protein, mRNA, or genomic DNA, such that the presence of HEMA protein, mRNA or genomic DNA is detected in the biological sample, and comparing the presence of HEMA protein, mRNA or genomic DNA in the control sample with the presence of HEMA protein, mRNA or genomic DNA in the test sample.

HEMA Nucleic Acids

Also provided in the invention are novel nucleic acids that include a nucleic acid sequence selected from the group consisting of HEMA, or its complement, as well as vectors and cells including these nucleic acids. Also provided are polypeptides encoded by HEMA nucleic acid or biologically active portions thereof.

Also included in the invention are nucleic acid fragments sufficient for use as hybridization probes to identify HEMA-encoding nucleic acids (e.g., HEMA mRNA) and fragments for use as polymerase chain reaction (PCR) primers for the amplification or mutation of HEMA nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated HEMA nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of any of HEMAS: 1-2 or a complement of any of these nucleotide sequences, can be isolated using standard molecular biology techniques and the sequence information provided herein. Using all or a portion of these nucleic acid sequences as a hybridization probe, HEMA nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to HEMA nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence shown in HEMAs: 1-2. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in HEMAs: 1-2 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of HEMA: 1-2 e.g., a fragment that can be used as a probe or primer or a fragment encoding a biologically active portion of HEMA. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a HEMA polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a HEMA polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human HEMA protein. Homologous nucleic acid sequences include those nucleic acid sequences that encode conservative amino acid substitutions (see below) in a HEMA polypeptide, as well as a polypeptide having a HEMA activity. A homologous amino acid sequence does not encode the amino acid sequence of a human HEMA polypeptide.

The nucleotide sequence determined from the cloning of human HEMA genes allows for the generation of probes and primers designed for use in identifying and/or cloning HEMA homologues in other cell types, e.g., from other tissues, as well as HEMA homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising a HEMA sequence, or an anti-sense strand nucleotide sequence of a nucleic acid comprising a HEMA sequence, or of a naturally occurring mutant of these sequences.

Probes based on human HEMA nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a HEMA protein, such as by measuring a level of a HEMA-encoding nucleic acid in a sample of cells from a subject e.g., detecting HEMA mRNA levels or determining whether a genomic HEMA gene has been mutated or deleted.

“A polypeptide having a biologically active portion of HEMA” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of HEMA” can be prepared by isolating a portion of HEMAs: 1-2, that encodes a polypeptide having a HEMA biological activity, expressing the encoded portion of HEMA protein (e.g., by recombinant expression in vitro) and assessing the activity of the encoded portion of HEMA. For example, a nucleic acid fragment encoding a biologically active portion of a HEMA polypeptide can optionally include an ATP-binding domain. In another embodiment, a nucleic acid fragment encoding a biologically active portion of HEMA includes one or more regions.

HEMA Variants

The invention further encompasses nucleic acid molecules that differ from the disclosed or referenced HEMA nucleotide sequences due to degeneracy of the genetic code. These nucleic acids thus encode the same HEMA protein as that encoded by nucleotide sequence comprising a HEMA nucleic acid as shown in, e.g., HEMA1-2

In addition to the rat HEMA nucleotide sequence shown in HEMAs: 1-2, it will be appreciated by those skilled in the art that DNA sequence polymorphisms that lead to changes in the amino acid sequences of a HEMA polypeptide may exist within a population (e.g., the human population). Such genetic polymorphism in the HEMA gene may exist among individuals within a population due to natural allelic variation. As used herein, the terms “gene” and “recombinant gene” refer to nucleic acid molecules comprising an open reading frame encoding a HEMA protein, preferably a mammalian HEMA protein. Such natural allelic variations can typically result in 1-5% variance in the nucleotide sequence of the HEMA gene. Any and all such nucleotide variations and resulting amino acid polymorphisms in HEMA that are the result of natural allelic variation and that do not alter the functional activity of HEMA are intended to be within the scope of the invention.

Moreover, nucleic acid molecules encoding HEMA proteins from other species, and thus that have a nucleotide sequence that differs from the human sequence of HEMA 1-2, are intended to be within the scope of the invention. Nucleic acid molecules corresponding to natural allelic variants and homologues of the HEMA DNAs of the invention can be isolated based on their homology to the human HEMA nucleic acids disclosed herein using the human cDNAs, or a portion thereof, as a hybridization probe according to standard hybridization techniques under stringent hybridization conditions. For example, a soluble human HEMA DNA can be isolated based on its homology to human membrane-bound HEMA. Likewise, a membrane-bound human HEMA DNA can be isolated based on its homology to soluble human HEMA.

Accordingly, in another embodiment, an isolated nucleic acid molecule of the invention is at least 6 nucleotides in length and hybridizes under stringent conditions to the nucleic acid molecule comprising the nucleotide sequence of HEMAs: 1-2. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding HEMA proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of HEMAs: 1-2 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of HEMAs: 1-2 or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of HEMAs: 1-2or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo et al., 1981, Proc Natl Acad Sci USA 78: 6789-6792.

Conservative Mutations

In addition to naturally-occurring allelic variants of the HEMA sequence that may exist in the population, the skilled artisan will further appreciate that changes can be introduced into an HEMA nucleic acid or directly into an HEMA polypeptide sequence without altering the functional ability of the HEMA protein. In some embodiments, the nucleotide sequence of HEMAs: 1 -2will be altered, thereby leading to changes in the amino acid sequence of the encoded HEMA protein. For example, nucleotide substitutions that result in amino acid substitutions at various “non-essential” amino acid residues can be made in the sequence of HEMAs: 1 -2A “non-essential” amino acid residue is a residue that can be altered from the wild-type sequence of HEMA without altering the biological activity, whereas an “essential” amino acid residue is required for biological activity. For example, amino acid residues that are conserved among the HEMA proteins of the present invention, are predicted to be particularly unamenable to alteration.

In addition, amino acid residues that are conserved among family members of the HEMA proteins of the present invention, are also predicted to be particularly unamenable to alteration. As such, these conserved domains are not likely to be amenable to mutation. Other amino acid residues, however, (e.g., those that are not conserved or only semi-conserved among members of the HEMA proteins) may not be essential for activity and thus are likely to be amenable to alteration.

Another aspect of the invention pertains to nucleic acid molecules encoding HEMA proteins that contain changes in amino acid residues that are not essential for activity. Such HEMA proteins differ in amino acid sequence from the amino acid sequences of polypeptides encoded by nucleic acids containing HEMAs: 1-2, yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%, and most preferably at least about 99% homologous to the amino acid sequence of the amino acid sequences of polypeptides encoded by nucleic acids comprising HEMAs: 1-2.

An isolated nucleic acid molecule encoding a HEMA protein homologous to can be created by introducing one or more nucleotide substitutions, additions or deletions into the nucleotide sequence of a nucleic acid comprising HEMAs: 1-2, such that one or more amino acid substitutions, additions or deletions are introduced into the encoded protein.

Mutations can be introduced into a nucleic acid comprising HEMAs: 1-2 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in HEMA is replaced with another amino acid residue from the same side chain family. Alternatively, in another embodiment, mutations can be introduced randomly along all or part of a HEMA coding sequence, such as by saturation mutagenesis, and the resultant mutants can be screened for HEMA biological activity to identify mutants that retain activity. Following mutagenesis of the nucleic acid, the encoded protein can be expressed by any recombinant technology known in the art and the activity of the protein can be determined.

In one embodiment, a mutant HEMA protein can be assayed for (1) the ability to form protein:protein interactions with other HEMA proteins, other cell-surface proteins, or biologically active portions thereof, (2) complex formation between a mutant HEMA protein and a HEMA ligand; (3) the ability of a mutant HEMA protein to bind to an intracellular target protein or biologically active portion thereof; (e.g., avidin proteins); (4) the ability to bind ATP; or (5) the ability to specifically bind a HEMA protein antibody.

In other embodiment, the fragment of the complementary polynucleotide sequence of HEMA 1-2 wherein the fragment of the complementary polynucleotide sequence hybridizes to the first sequence.

In other specific embodiments, the nucleic acid is RNA or DNA. The fragment or the fragment of the complementary polynucleotide sequence of HEMA 1-2, wherein the fragment is between about 10 and about 100 nucleotides in length, e.g., between about 10 and about 90 nucleotides in length, or about 10 and about 75 nucleotides in length, about 10 and about 50 bases in length, about 10 and about 40 bases in length, or about 15 and about 30 bases in length.

Antisense

Another aspect of the invention pertains to isolated antisense nucleic acid molecules that are hybridizable to or complementary to the nucleic acid molecule comprising the nucleotide sequence of a HEMA sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire HEMA coding strand, or to only a portion thereof. Nucleic acid molecules encoding fragments, homologs, derivatives and analogs of a HEMA protein, or antisense nucleic acids complementary to a nucleic acid comprising a HEMA nucleic acid sequence are additionally provided.

In one embodiment, an antisense nucleic acid molecule is antisense to a “coding region” of the coding strand of a nucleotide sequence encoding HEMA. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding HEMA. The term “noncoding region” refers to 5′ and 3′ sequences which flank the coding region that are not translated into amino acids (i.e., also referred to as 5′ and 3′ untranslated regions).

Given the coding strand sequences encoding HEMA disclosed herein, antisense nucleic acids of the invention can be designed according to the rules of Watson and Crick or Hoogsteen base pairing. The antisense nucleic acid molecule can be complementary to the entire coding region of HEMA mRNA, but more preferably is an oligonucleotide that is antisense to only a portion of the coding or noncoding region of HEMA mRNA. For example, the antisense oligonucleotide can be complementary to the region surrounding the translation start site of HEMA mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (ie., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a HEMA protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual 5-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave HEMA mRNA transcripts to thereby inhibit translation of HEMA mRNA. A ribozyme having specificity for a HEMA-encoding nucleic acid can be designed based upon the nucleotide sequence of a HEMA DNA disclosed herein. For example, a derivative of a Tetrahymena L-19 IVS RNA can be constructed in which the nucleotide sequence of the active site is complementary to the nucleotide sequence to be cleaved in a HEMA-encoding mRNA. See, e.g., Cech et al. U.S. Pat. No. 4,987,071; and Cech et al. U.S. Pat. No. 5,116,742. Alternatively, HEMA mRNA can be used to select a catalytic RNA having a specific ribonuclease activity from a pool of RNA molecules. See, e.g., Bartel et al., (1993) Science 261:1411-1418.

Alternatively, HEMA gene expression can be inhibited by targeting nucleotide sequences complementary to the regulatory region of a HEMA nucleic acid (e.g., the HEMA promoter and/or enhancers) to form triple helical structures that prevent transcription of the HEMA gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of HEMA can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of HEMA can be used in therapeutic and diagnostic applications. For example, PNAs can be used as antisense or antigene agents for sequence-specific modulation of gene expression by, e.g., inducing transcription or translation arrest or inhibiting replication. PNAs of HEMA can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of HEMA can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of HEMA can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

HEMA Polypeptides

One aspect of the invention pertains to isolated HEMA proteins, and biologically active portions thereof, or derivatives, fragments, analogs or homologs thereof. Also provided are polypeptide fragments suitable for use as immunogens to raise anti-HEMA antibodies. In one embodiment, native HEMA proteins can be isolated from cells or tissue sources by an appropriate purification scheme using standard protein purification techniques. In another embodiment, HEMA proteins are produced by recombinant DNA techniques. Alternative to recombinant expression, a HEMA protein or polypeptide can be synthesized chemically using standard peptide synthesis techniques.

An “isolated” or “purified” protein or biologically active portion thereof is substantially free of cellular material or other contaminating proteins from the cell or tissue source from which the HEMA protein is derived, or substantially free from chemical precursors or other chemicals when chemically synthesized. The language “substantially free of cellular material” includes preparations of HEMA protein in which the protein is separated from cellular components of the cells from which it is isolated or recombinantly produced. In one embodiment, the language “substantially free of cellular material” includes preparations of HEMA protein having less than about 30% (by dry weight) of non-HEMA protein (also referred to herein as a “contaminating protein”), more preferably less than about 20% of non-HEMA protein, still more preferably less than about 10% of non-HEMA protein, and most preferably less than about 5% non-HEMA protein. When the HEMA protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of HEMA protein in which the protein is separated from chemical precursors or other chemicals that are involved in the synthesis of the protein. In one embodiment, the language “substantially free of chemical precursors or other chemicals” includes preparations of HEMA protein having less than about 30% (by dry weight) of chemical precursors or non-HEMA chemicals, more preferably less than about 20% chemical precursors or non-HEMA chemicals, still more preferably less than about 10% chemical precursors or non-HEMA chemicals, and most preferably less than about 5% chemical precursors or non-HEMA chemicals.

Biologically active portions of a HEMA protein include peptides comprising amino acid sequences sufficiently homologous to or derived from the amino acid sequence of the HEMA protein, e.g., the amino acid sequence encoded by a nucleic acid comprising HEMA 1-20 that include fewer amino acids than the full length HEMA proteins, and exhibit at least one activity of a HEMA protein. Typically, biologically active portions comprise a domain or motif with at least one activity of the HEMA protein. A biologically active portion of a HEMA protein can be a polypeptide which is, for example, 10, 25, 50, 100 or more amino acids in length.

A biologically active portion of a HEMA protein of the present invention may contain at least one of the above-identified domains conserved between the HEMA proteins. An alternative biologically active portion of a HEMA protein may contain at least two of the above-identified domains. Another biologically active portion of a HEMA protein may contain at least three of the above-identified domains. Yet another biologically active portion of a HEMA protein of the present invention may contain at least four of the above-identified domains.

Moreover, other biologically active portions, in which other regions of the protein are deleted, can be prepared by recombinant techniques and evaluated for one or more of the functional activities of a native HEMA protein.

In some embodiments, the HEMA protein is substantially homologous to one of these HEMA proteins and retains its the functional activity, yet differs in amino acid sequence due to natural allelic variation or mutagenesis, as described in detail below.

In specific embodiments, the invention includes an isolated polypeptide comprising an amino acid sequence that is 80% or more identical to the sequence of a polypeptide whose expression is modulated in a mammal to which PPARγ ligand is administered.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence comprising HEMAS: 1-15.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

HEMA Chimeric and Fusion Proteins

The invention also provides HEMA chimeric or fusion proteins. As used herein, a HEMA “chimeric protein” or “fusion protein” comprises an HEMA polypeptide operatively linked to a non-HEMA polypeptide. A “HEMA polypeptide” refers to a polypeptide having an amino acid sequence corresponding to HEMA, whereas a “non-HEMA polypeptide” refers to a polypeptide having an amino acid sequence corresponding to a protein that is not substantially homologous to the HEMA protein, e.g., a protein that is different from the HEMA protein and that is derived from the same or a different organism. Within an HEMA fusion protein the HEMA polypeptide can correspond to all or a portion of an HEMA protein. In one embodiment, an HEMA fusion protein comprises at least one biologically active portion of an HEMA protein. In another embodiment, an HEMA fusion protein comprises at least two biologically active portions of an HEMA protein. In yet another embodiment, an HEMA fusion protein comprises at least three biologically active portions of an HEMA protein. Within the fusion protein, the term “operatively linked” is intended to indicate that the HEMA polypeptide and the non-HEMA polypeptide are fused in-frame to each other. The non-HEMA polypeptide can be fused to the N-terminus or C-terminus of the HEMA polypeptide.

For example, in one embodiment an HEMA fusion protein comprises an HEMA domain operably linked to the extracellular domain of a second protein. Such fusion proteins can be further utilized in screening assays for compounds which modulate HEMA activity (such assays are described in detail below).

In yet another embodiment, the fusion protein is a GST-HEMA fusion protein in which the HEMA sequences are fused to the C-terminus of the GST (i.e., glutathione S-transferase) sequences. Such fusion proteins can facilitate the purification of recombinant HEMA.

In another embodiment, the fusion protein is an HEMA protein containing a heterologous signal sequence at its N-terminus. For example, a native HEMA signal sequence can be removed and replaced with a signal sequence from another protein. In certain host cells (e.g., mammalian host cells), expression and/or secretion of HEMA can be increased through use of a heterologous signal sequence.

In yet another embodiment, the fusion protein is an HEMA-immunoglobulin fusion protein in which the HEMA sequences comprising one or more domains are fused to sequences derived from a member of the immunoglobulin protein family. The HEMA-immunoglobulin fusion proteins of the invention can be incorporated into pharmaceutical compositions and administered to a subject to inhibit an interaction between a HEMA ligand and a HEMA protein on the surface of a cell, to thereby suppress HEMA-mediated signal transduction in vivo. The HEMA-immunoglobulin fusion proteins can be used to affect the bioavailability of an HEMA cognate ligand. Inhibition of the HEMA ligand/HEMA interaction may be useful therapeutically for both the treatments of proliferative and differentiative disorders, as well as modulating (e.g. promoting or inhibiting) cell survival. Moreover, the HEMA-immunoglobulin fusion proteins of the invention can be used as immunogens to produce anti-HEMA antibodies in a subject, to purify HEMA ligands, and in screening assays to identify molecules that inhibit the interaction of HEMA with a HEMA ligand.

An HEMA chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An HEMA-encoding nucleic acid can be cloned into such an expression vector such that the fusion moiety is linked in-flame to the HEMA protein.

HEMA Agonists and Antagonists

The present invention also pertains to variants of the HEMA proteins that function as either HEMA agonists (mimetics) or as HEMA antagonists. Variants of the HEMA protein can be generated by mutagenesis, e.g., discrete point mutation or truncation of the HEMA protein. An agonist of the HEMA protein can retain substantially the same, or a subset of, the biological activities of the naturally occurring form of the HEMA protein. An antagonist of the HEMA protein can inhibit one or more of the activities of the naturally occurring form of the HEMA protein by, for example, competitively binding to a downstream or upstream member of a cellular signaling cascade which includes the HEMA protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the HEMA proteins.

Variants of the HEMA protein that function as either HEMA agonists (mimetics) or as HEMA antagonists can be identified by screening combinatorial libraries of mutants, e.g., truncation mutants, of the HEMA protein for HEMA protein agonist or antagonist activity. In one embodiment, a variegated library of HEMA variants is generated by combinatorial mutagenesis at the nucleic acid level and is encoded by a variegated gene library. A variegated library of HEMA variants can be produced by, for example, enzymatically ligating a mixture of synthetic oligonucleotides into gene sequences such that a degenerate set of potential HEMA sequences is expressible as individual polypeptides, or alternatively, as a set of larger fusion proteins (e.g., for phage display) containing the set of HEMA sequences therein. There are a variety of methods which can be used to produce libraries of potential HEMA variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential HEMA sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the HEMA protein coding sequence can be used to generate a variegated population of HEMA fragments for screening and subsequent selection of variants of an HEMA protein. In one embodiment, a library of coding sequence fragments can be generated by treating a double stranded PCR fragment of a HEMA coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the HEMA protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of HEMA proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify HEMA variants (Arkin and Yourvan (1992) PNAS 89:7811-7815; Delgrave et al. (1993) Protein Engineering 6:327-331).

Anti-HEMA Antibodies

An isolated HEMA protein, or a portion or fragment thereof, can be used as an immunogen to generate antibodies that bind HEMA using standard techniques for polyclonal and monoclonal antibody preparation. The full-length HEMA protein can be used or, alternatively, the invention provides antigenic peptide fragments of HEMA for use as immunogens. The antigenic peptide of HEMA comprises at least 8 amino acid residues of the amino acid sequence encoded by a nucleic acid comprising the nucleic acid sequence shown in HEMAS:1-2 and encompasses an epitope of HEMA such that an antibody raised against the peptide forms a specific immune complex with HEMA. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of HEMA that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

HEMA polypeptides or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen. Such antibodies include, but are not limited to, polyclonal, monoclonal, chimeric, single chain, F_{ab} and F_{(ab′)2} fragments, and an F_{ab} expression library. Various procedures known within the art may be used for the production of polyclonal or monoclonal antibodies to an HEMA protein sequence, or derivatives, fragments, analogs or homologs thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed HEMA protein or a chemically synthesized HEMA polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against HEMA can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of HEMA. A monoclonal antibody composition thus typically displays a single binding affinity for a particular HEMA protein with which it immunoreacts. For preparation of monoclonal antibodies directed towards a particular HEMA protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

According to the invention, techniques can be adapted for the production of single-chain antibodies specific to a HEMA protein (see e.g., U.S. Pat. No. 4,946,778). In addition, methods can be adapted for the construction of F_{ab} expression libraries (see e.g., Huse, et al, 1989 Science 246: 1275-1281) to allow rapid and effective identification of monoclonal F_{ab} fragments with the desired specificity for a HEMA protein or derivatives, fragments, analogs or homologs thereof. Non-human antibodies can be “humanized” by techniques well known in the art. See e.g., U.S. Pat. No. 5,225,539. Antibody fragments that contain the idiotypes to a HEMA protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-HEMA antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst. 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a HEMA protein is facilitated by generation of hybridomas that bind to the fragment of a HEMA protein possessing such a domain. Antibodies that are specific for one or more domains within a HEMA protein, e.g., domains spanning the above-identified conserved regions of HEMA family proteins, or derivatives, fragments, analogs or homologs thereof, are also provided herein.

Anti-HEMA antibodies may be used in methods known within the art relating to the localization and/or quantitation of a HEMA protein (e.g., for use in measuring levels of the HEMA protein within appropriate physiological samples, for use in diagnostic methods, for use in imaging the protein, and the like). In a given embodiment, antibodies for HEMA proteins, or derivatives, fragments, analogs or homologs thereof, that contain the antibody derived binding domain, are utilized as pharmacologically-active compounds [hereinafter “Therapeutics”].

An anti-HEAM antibody (e.g., monoclonal antibody) can be used to isolate HEMA by standard techniques, such as affinity chromatography or immunoprecipitation. An anti-HEMA antibody can facilitate the purification of natural HEMA from cells and of recombinantly produced HEMA expressed in host cells. Moreover, an anti-HEMA antibody can be used to detect HEMA protein (e.g., in a cellular lysate or cell supernatant) in order to evaluate the abundance and pattern of expression of the HEMA protein. Anti-HEMA antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

HEMA Recombinant Expression Vectors and Host Cells

Another aspect of the invention pertains to vectors, preferably expression vectors, containing a nucleic acid encoding HEMA protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., HEMA proteins, mutant forms of HEMA, fusion proteins, etc.).

The recombinant expression vectors of the invention can be designed for expression of HEMA in prokaryotic or eukaryotic cells. For example, HEMA can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the HEMA expression vector is a yeast expression vector. Examples of vectors for expression in yeast S. cerevisiae include pYepSec 1 (Baldari, et al., (1987) EMBO J 6:229-234), pMFa (Kuijan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, HEMA can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banedji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to HEMA mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, HEMA protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding HEMA or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an HEMA protein. Accordingly, the invention further provides methods for producing HEMA protein using the host cells of the invention. In one embodiment, the method comprises culturing the host cell of invention (into which a recombinant expression vector encoding HEMA has been introduced) in a suitable medium such that HEMA protein is produced. In another embodiment, the method further comprises isolating HEMA from the medium or the host cell.

Kits and Nucleic Acid Collections for Identifying HEMA Nucleic Acids

In another aspect, the invention provides a kit useful for examining a pathophysiology associated with a PPARγ-mediated pathway. The kit can include nucleic acids that detect two or more HEMA sequences. In preferred embodiments, the kit includes reagents which detect 3, 4, 5, 6, 8, 10, 12, 15, 20, 25, 30, 35,40 or all of the HEMA nucleic acid sequences.

The invention also includes an isolated plurality of sequences which can identify one or more HEMA responsive nucleic acid sequences.

The kit or plurality may include, e.g., sequence homologous to HEMA nucleic acid sequences, or sequences which can specifically identify one or more HEMA nucleic acid sequences.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of assessing hematopoietic status in a subject, the method comprising:
(a) providing a test cell population from the subject, wherein at least one cell in the test cell population is capable of expressing one or more nucleic acid sequences selected from the group consisting of HEMA 1-39 and 40;
(b) measuring the expression of one or more of the nucleic acid sequences in said test cell population; and
(c) comparing the expression of the nucleic acid sequences in the test cell population to the expression of the nucleic acid sequences in a reference cell population comprising at least one cell whose hematopoietic status is known, thereby indicating hematopoietic status in the subject.

2. The method of claim 1, wherein the method comprises comparing the expression of five or more of the nucleic acid sequences.

3 The method of claim 1, wherein the method comprises comparing the expression of 20 or more of the nucleic acid sequences.

4. The method of claim 1, wherein the method comprises comparing the expression of 25 or more of the nucleic acid sequences.

5. The method of claim 1, wherein the expression of the nucleic acid sequences in the test cell population is increased as compared to the reference cell population.

6. The method of claim 1, wherein the test cell population is provided in vitro.

7. The method of claim 1, wherein the test cell population is provided ex vivo from a mammalian subject.

8. The method of claim 1, wherein the test cell is provided in vivo in a mammalian subject.

9. The method of claim 1, wherein the test cell population is derived from a human or rodent subject.

10. The method of claim 1, wherein the test cell includes a hematopoietic cell.

11. A method of diagnosing or determining the susceptibility to a hematopoietic disorder in a subject, the method comprising:
(a) providing from the subject a test cell population comprising cells capable of expressing one or more nucleic acid sequences selected from the group consisting of HEMA 1-39 and 40;
(b) measuring expression of one or more of the nucleic acid sequences in the test cell population; and
(c) comparing the expression of the nucleic acid sequences in the test cell population to the expression of the nucleic acid sequences in a reference cell population comprising at least one cell from a subject not suffering from a hematopoietic disorder; and
(d) identifying a difference in expression levels of the nucleic acid sequences, if present, in the test cell population and reference cell population,
thereby diagnosing or determining the susceptibility to a hematopoietic disorder in the subject.

12. The method of claim 11, wherein the hematopoietic disorder is selected from the group consisting of anemia, leukemia and lymphoma.

13. A method of treating a hematopoietic disorder in a subject, the method comprising administering to the subject an agent that modulates the expression or the activity of one or more nucleic acids selected from the group consisting of HEMA 1-39 and 40

14. The method of claim 13, wherein the agent is a peptide, peptidomimetic, small molecule or other drug.

15. A method of assessing the efficacy of a treatment of a hematopoietic disorder in a subject, the method comprising:
(a) providing from the subject a test cell population comprising cells capable of expressing one or more nucleic acid sequences selected from the group consisting of HEMA 1-39 and 40;
(b) detecting expression of one or more of the nucleic acid sequences in the test cell population;
(c) comparing the expression of the nucleic acid sequences in the test cell population to the expression of the nucleic acid sequences in a reference cell population comprising at least one cell from a subject not suffering a hematopoietic disorder; and
(e) identifying a difference in expression levels of the nucleic acid sequences, if present, in the test cell population and reference cell population, thereby assessing the efficacy of treatment of the hematopoietic disorder in the subject.

16. An isolated nucleic acid molecule encoding a polypeptide comprising an amino acid sequence that is at least 75% identical to SEQ ID NO: 2, 4, or 5 or the complement of the nucleic acid molecule.

17. A nucleic acid vector comprising the nucleic acid molecule of claim 16.

18. A host cell comprising the isolated nucleic acid molecule of claim 16.

19. An isolated polypeptide at least 80% identical to a polypeptide selected from the group consisting of:
a) a polypeptide comprising an amino acid sequence of SEQ. ID NO:2, 4 or 5;
b) a fragment of a polypeptide comprising an amino acid sequence of SEQ ID NO: 2, 4 or 5, wherein the fragment comprises at least 6 contiguous amino acids of SEQ ID NO: 2, 4 or 5 ;
c) a derivative of a polypeptide comprising an amino acid sequence of SEQ ID NO: 2, 4 or 5
d) an analog of a polypeptide comprising an amino acid sequence of SEQ ID NO: 2, 4 or 5; and
e) a homolog of a polypeptide comprising an amino acid sequence of SEQ ID NO: 2, 4 or 5;

20. An antibody that selectively binds to the polypeptide of claim 19, and fragments, homologs, analogs and derivatives of the antibody.

21. A pharmaceutical composition comprising the nucleic acid of claim 16.

22. A pharmaceutical composition comprising the polypeptide of claim 19.

23. A method of detecting the presence of the polypeptide of claim 19 in a sample, comprising contacting the sample with a compound that selectively binds to the polypeptide of claim 19 and determining whether the compound bound to the polypeptide of claim 19 is present in the sample.

24. A method for modulating the activity of the polypeptide of claim 19, the method comprising contacting a cell sample comprising the polypeptide of claim 19 with a compound that binds to said polypeptide in an amount sufficient to modulate the activity of the polypeptide.

25. A method of promoting migration of a hematopoietic stem cell, the method comprising contacting the hematopoietic stem cell with the polypeptide of claim 19 in an amount sufficient to promote migration of the hematopoietic stem cell.

26. The method of claim 25, wherein the hematopoietic stem cell is a bone marrow cell or a fetal liver cell.

27. A method of inhibiting proliferation or differentiation of a hematopoietic stem cell or a endothelial cell, the method comprising contacting the cell with the polypeptide of claim 19 in an amount sufficient to inhibit proliferation of the cell.

28. The method of claim 27, wherein the hematopoietic stem cell or the endothelial cell is provided in vitro.

28. The method of claim 27, wherein the hematopoietic stem cell or the endothelial cell is provided ex vivo from a mammalian subject.

30. The method of claim 27, wherein the hematopoietic stem cell or the endothelial cell is provided in vivo in a mammalian subject.

31. The method of claim 27, wherein the hematopoietic stem cell or the endothelial cell is derived from a human or rodent subject.

32. A method of identifying an agent that modulates hematopoiesis, the method comprising
(a) contacting the polypeptide of claim 19 and a test agent; and
(b) detecting a complex between the polypeptide the agent, wherein the presence of the complex indicates that the agent modulates hematopoiesis.

33. A method of identifying an agent that modulates hematopoiesis, the method comprising
(a) providing a hematopoietic stem cell;
(b) contacting the hematopoietic stem cell with the polypeptide of claim 19 and a test agent; and
(c) comparing the proliferation of the hematopoietic stem cell in the presence of the polypeptide and the test agent to the proliferation of the hematopoietic stem cell in the absence of the test agent,
wherein an alteration in the proliferation or differentiation of the hematopoietic stem cell in the presence of the test agent compared to the proliferation or differentiation of the hematopoietic stem cell in the absence of the test agent indicates the test agent modulates hematopoiesis.

34. A chimeric polypeptide comprising a first domain and a second domain linked by a covalent bond, the first domain comprising a chemokine, and the second domain comprising a hematopoietic modulating sequence.

35 The polypeptide of claim 34, wherein the chemokine is a CXC or CC chemokine.

36. The polypeptide of claim 34, wherein the hematopoietic modulating sequence comprises the amino acid sequence of SEQ ID NO: 6.

37. An isolated nucleic acid molecule encoding a polypeptide of claim 34.

38. A nucleic acid vector comprising the nucleic acid molecule of claim 37.

39. A host cell comprising the isolated nucleic acid molecule of claim 37.

40. A pharmaceutical composition comprising the nucleic acid of claim 37.

41. An antibody that selectively binds to the polypeptide of claim 34, and fragments, homologs, analogs and derivatives of the antibody.

42. A pharmaceutical composition comprising the polypeptide of claim 34.

43. A method of detecting the presence of the polypeptide of claim 34 in a sample, comprising contacting the sample with a compound that selectively binds to the polypeptide of claim 32 and determining whether the compound bound to the polypeptide of claim 32 is present in the sample.

44. A method for modulating the activity of the polypeptide of claim 34, the method comprising contacting a cell sample comprising the polypeptide of claim 34 with a compound that binds to said polypeptide in an amount sufficient to modulate the activity of the polypeptide.

45. A method of promoting migration of a hematopoietic stem cell, the method comprising contacting the hematopoietic stem cell with the polypeptide of claim 34 in an amount sufficient to promote migration of the hematopoietic stem cell.

46. The method of claim 45, wherein the hematopoietic stem cell is a bone marrow cell or a fetal liver cell.

47. A method of inhibiting proliferation or differentiation of a hematopoietic stem cell or endothelial cell, the method comprising contacting the cell with the polypeptide of claim 32 in an amount sufficient to inhibit proliferation of the cell.

48. The method of claim 47, wherein the hematopoietic stem cell or endothelial cell is provided in vitro.

49. The method of claim 47, wherein the hematopoietic stem cell or endothelial cell is provided ex vivo from a mammalian subject.

50. The method of claim 47, wherein the hematopoietic stem cell or endothelial cell is provided in vivo in a mammalian subject.

51. The method of claim 47, wherein the hematopoietic stem cell is derived from a human or rodent subject.

52. A method of identifying an agent that modulates hematopoiesis, the method comprising
(a) contacting the polypeptide of claim 34 and a test agent; and
(b) detecting a complex between the polypeptide the agent, wherein the presence of the complex indicates that the agent modulates hematopoiesis.

53. A method of identifying an agent that modulates hematopoiesis, the method comprising
(a) providing a hematopoietic stem cell;
(b) contacting the hematopoietic stem cell with the polypeptide of claim 34 and a test agent; and
(c) comparing the proliferation or differentiation of the hematopoietic stem cell in the presence of the polypeptide and the test agent to the proliferation or differentiation of the hematopoietic stem cell in the absence of the test agent,
wherein an alteration in the proliferation or differentiation of the hematopoietic stem cell in the presence of the test agent compared to the proliferation or differentiation of the hematopoietic stem cell in the absence of the test agent indicates the test agent modulates hematopoiesis.

54. A kit which detects two or more of the nucleic acid sequences selected from the group consisting of HEMA 1-39 and 40.

55. An array which detects one or more of the nucleic acid selected from the group consisting of HEMA 1-39 and 40.

56. A plurality of nucleic acid comprising one or more of the nucleic acid selected from the group consisting of HEMA1-39 and 40.
